(19) United States
(12) Patent Application Publication
(10) Pub. No.: US 2010/0086655 A1
(43) Pub. Date: Apr. 8, 2010

(54) PROCESS OF SELECTING A PREPARATION METHOD, A PACKAGING AND SHIPPING METHOD, OR OTHER DISPOSTION OF A FOODSTUFF, AND PROCESS OF DETERMINING IF A FOODSTUFF IS FRESH OR HAS PREVIOUSLY BEEN FROZEN

(76) Inventor: MICHAEAL G SINGER, Harrisville, MI (US)

Correspondence Address: WEINER & BURT, P.C., 635 N US-23, POB 186, HARRISVILLE, MI, 48740, US

(21) Appl. No.: 12/632,771

(22) Filed: Dec. 7, 2009

Related U.S. Application Data
(63) Continuation-in-part of application No. 12/600,267.
(60) Provisional application No. 60/939,703, filed on May 23, 2007.

(51) Int. Cl. G01N 33/02

(52) U.S. Cl. 426/232

(57) ABSTRACT

A process of determining if a foodstuff is fresh or has previously been frozen. The foodstuff is subjected to bioelectrical impedance analysis including measurement or calculation of values of resistance, reactance, impedance, capacitance, and/or phase angle. The results illustrate whether or not the foodstuff has been previously frozen or is fresh. Also provided is a process of selecting a preparation method, a packaging and shipping method, or disposition of a foodstuff. First determining a palatability value for the foodstuff. Second determining as a function of the determined palatability value whether or not the foodstuff is to be prepared for consumption, is to be packaged and shipped, or is to be disposed of.

The present application is a continuation-in-part of U.S. patent application Ser. No. 12/600,267 filed Nov. 15, 2009, International Patent Application PCT/US08/64696 filed May 23, 2008, U.S. Provisional patent application 60/939,703 filed May 23, 2007, and U.S. patent application Ser. No. 11/912,887 filed Oct. 27, 2007, the entire contents of which applications are incorporated herein by reference thereto.

### BACKGROUND OF THE INVENTION

The present invention relates generally to a process of selecting a preparation method, a packaging and shipping method, or disposition of a foodstuff, and to a process of determining whether or not a foodstuff is fresh or has previously been frozen,

After safety (which is often assumed), consumers believe palatability to be the single most important component of meat and foodstuff quality. Contamination of foodstuff has become a significant threat to public health.

Whether the source of the contamination is infection or pollutant, a cumulative threshold of effect is attained which results in morbidity and mortality to the consuming entity. A common aspect of the effects of these contaminants is the impact upon the cellular architecture, especially the membrane, of the biological foodstuff.

Live and previously-live foodstuffs have in common a basic cellular structure that applicant has previously demonstrated relates to freshness and palatability.

The prior, but not necessarily relevant, art is exemplified in the following U.S. patents and patent applications.

U.S. Pat. No. 4,985,125, issued in 1991, to Watanabe, et al; entitled; “Method for Detecting Meat Freshness Using a Biosensor”, in which is described; “A biosensor comprising a main body and a built-in sensing electrode provided therein, wherein the sensing electrode is provided with a sensing part to which a buffer solution can be constantly fed and the main body is provided with an inlet which is brought into direct contact with a specimen to thereby incorporate the molecule to be assayed from the specimen there through, the inlet being covered with a membrane through which the molecule to be assayed can permeate. This biosensor enables a specified molecule contained in a specimen to be rapidly and conveniently assayed without requiring any pretreatment of the specimen.”

U.S. Pat. No. 5,088,822, issued in 1992 to Kanda, entitled; “Meat Freshness Measuring Apparatus”, in which is described; “A meat freshness measuring apparatus measures the freshness of meat by detecting changes in a pigment contained in the meat. For this purpose, the meat is exposed to rays of light of different wavelengths applied from a light source, and the rays obtained from the meat are separated into spectra for the respective wavelengths, which are received by a photoelectric conversion element. Spectrum data for each wavelength are amplified and then the amplified data are converted to a digital signal by an A/D converter, whereby the digital signal is stored in a RAM. Based on the stored spectrum data and using a prescribed equation of calculation, a content of the pigment in the meat is calculated and outputted by a CPU.

U.S. Pat. No. 5,788,643, issued in 1998 to Feldman entitled “Process for Monitoring Patients with Chronic Congestive Heart Failure”, in which is described “In a process for monitoring patients with chronic congestive heart failure, a high frequency current is passed between electrodes applied to two limbs of a patient. The current, voltage and phase angle between the measured current and voltage are measured to enable the calculation of congestive heart failure (CHF) indicia values. The calculated CHF indicia values are then compared with baseline values established when the patient is in a known, stable condition. Intervention is initiated if the differences between the calculated CHF indicia values and the baseline values are outside of established tolerances. The CHF indicia values may include resistance, reactance, impedance, total body water and extracellular water. Moreover, the CHF indicia values may include a figure of merit indicative of the hydration status of the patient.

U.S. Published Patent Application 2004/0014235, published in 2004 by Kelly, et al, entitled “Polymeric Food Spoilage Sensor”, in which is described, “A polymeric food spoilage sensor comprises a polymer containing a polyazamacrocyclic transition metal complex. The complex selectively binds biogenic amines, such as cadaverine, putrescine and histamine, which are released by food spoilage microorganisms. The polymer undergoes a detectable color change upon exposure to biogenic amine, thus indicating that food spoilage has probably occurred. In one embodiment, the polymer is molecularly imprinted with the biogenic amine to impart selective binding affinity. The polymer is easily incorporated in common food containers and can be employed in fiber optic detection devices.”

U.S. Published Patent Application 2005/0058751, published in 2005 by Brotsky et al., entitled; “Yield and Shelf Life for Meats”, in which is described an; “The invention is directed to a process for treating meat to achieve higher yield, without negatively affecting the appearance of the meat. The process comprises the steps of 1) treating the meat, preferably by injection, with a higher pH phosphate solution having a pH of above pH 6.0 and 2) after treating the meat with the higher pH phosphate solution, contacting the meat, preferably by dipping or spraying, with a lower pH solution of preferably less than about pH 5.6.

The achieved goal of the present invention is to avoid the disadvantages and deficiencies of conventional methods and techniques, and to provide novel methods for determination of the safety for consumption of foodstuffs.

### SUMMARY OF THE INVENTION

The present invention provides a process of selecting a preparation method, a packaging and shipping method, or disposition of a foodstuff, comprising the steps of: first determining a palatability value for the foodstuff; and second determining as a function of the determined palatability value whether or not the foodstuff is to be prepared for consumption, is to be packaged and shipped, or is to be disposed of.

The present invention also provides a process of determining whether or not a foodstuff is fresh or has previously been frozen, comprising the steps of: subjecting the foodstuff to bioelectrical impedance analysis including measurement and/or calculation of values of resistance, reactance, impedance, capacitance, and/or phase angle of the foodstuff; and utilizing results of the subjecting step to illustrate whether or not the foodstuff has been previously frozen or is fresh.

An object of the present invention is to provide a process as described hereinabove, including the steps of: determining a first palatability index of the foodstuff at harvesting the foodstuff or as close in time as possible to the harvesting of the foodstuff; determining a second palatability index of the foodstuff just prior to the second determining step or as close in time as possible to performing the second determining step; and determining the palatability value as a function of the first and second palatability indices.

Another object of the present invention is to provide a process as described hereinabove, including selecting a method of preparation for the foodstuff in dependence on the determined palatability value.

Another object of the present invention is to provide a process as described hereinabove, including preparing the foodstuff in accordance with the selected method to optimize the determined palatability value.

Another object of the present invention is to provide a process as described hereinabove, including selecting of a method of preparation for the foodstuff in dependence on the determined palatability value comprises grading of the determined palatability value for preparation of the foodstuff as an entree rather than as a part of a sauced, casserole, stew and/or secondary or supporting portion of a meal.

Another object of the present invention is to provide a process as described hereinabove, using the determined palatability value as a means to determine packaging type and transportation rate in that at the point of assessment the foodstuff is sorted by palatability with application and value in mind, and then packed and sent to distribution or end-user points based upon the determined palatability value.

Another object of the present invention is to provide a process as described hereinabove, wherein the first determining step includes subjecting the foodstuff to bioelectrical impedance analysis including measurement and/or calculation of values of resistance, reactance, impedance, capacitance, and/or phase angle of the foodstuff.

Another object of the present invention is to provide a process as described hereinabove, utilizing results of the subjecting step to illustrate whether or not the foodstuff has been previously frozen or is fresh.

Another object of the present invention is to provide a process as described hereinabove, wherein when the reactance value is zero or nearly zero, this indicates that the foodstuff has previously been frozen.

Another object of the present invention is to provide a process as described hereinabove, wherein the phase angle is used to indicate whether or not the foodstuff has been previously frozen or is fresh.

Another object of the present invention is to provide a process as described hereinabove, wherein determining of the first and second palatability indices includes subjecting the foodstuff to bioelectrical impedance analysis including measurement and/or calculation of values of resistance, reactance, impedance, capacitance, and/or phase angle of the foodstuff.

Another object of the present invention is to provide a process as described hereinabove, including utilizing results of the subjecting step to illustrate whether or not the foodstuff has been previously frozen or is fresh.

Another object of the present invention is to provide a process as described hereinabove, wherein when the reactance value is zero or nearly zero, this indicates that the foodstuff has previously been frozen.

Another object of the present invention is to provide a process as described hereinabove, wherein the phase angle is used to indicate whether or not the foodstuff has been previously frozen or is fresh.

Another object of the present invention is to provide a process as described hereinabove, wherein when the reactance value is zero or nearly zero, this indicates that the foodstuff has previously been frozen.

Other objects, advantages, and features of the present invention will become apparent to those persons skilled in this particular area of technology and to other persons after having been exposed to the present patent application.

### DETAILED DESCRIPTION OF THE INVENTION

Contamination of foodstuff has become a significant threat to public health. Whether the source of the contamination is infection or pollutant, a cumulative threshold of effect is attained which results in morbidity and mortality to the consuming entity.

A common aspect of the effects of these contaminants is the impact upon the cellular architecture, especially the membrane, of the biological foodstuff. Live and previously-live foodstuffs have in common a basic cellular structure which applicant has previously demonstrated relates to freshness and palatability.

The present invention is based upon the electrical illustration of that cellular level of physiology (pathophysiology) through impedance analysis in which applicant utilizes the measured electrical values of impedance, resistance, and reactance and calculate phase angle and capacitance and track them over time to illustrate freshness and palatability.

The measurement of the impedance may be done at various frequencies, power levels, constant current strengths and electrode configurations with the resultant basis of establishing a ‘normal’ value and tracking episodically and/or serially the changes associated with freshness, palatability and safety of the foodstuff.

In the present invention, it is specifically the difference in the rate of change from established averages of freshness and palatability which are indicative of contamination from any source as the level of the contamination reaches a zenith through in the case of a biological (bacterial) its colony count or in that of a pollutant its accumulated concentration is so great as to adversely impact the cellular architecture (process and membranes) as evidenced by an increased rate of change of the measured electrical values greater than that associated with changes of freshness and palatability.

As the rate of change accelerates from its normal pace, it is indicative of an adverse condition for the foodstuff consumer which may warrant further testing or simply the disposal of the foodstuff.

Thus, the present invention provides a method and system to obtain and use the measured values and products of bioelectrical impedance analysis (BIA) as an objective means to equivalently illustrate electrically, various physiological characteristics, and upon which characterization the safety of foodstuffs for consumption can be objectively described and compared and practically utilized.

The method of BIA measurement may comprise various configurations so as to accommodate the diversity of foodstuffs so measured to the extent that the interface with the foodstuff (electrode array/scheme, electrical power management (frequencies, current and voltages)) and circuit models (series and/or parallel) may be varied as such to incorporate the subject foodstuff within the controlled electrical circuit or field of the BIA measurement comprised in such manner as to complete the measurement.

The interfaces for electrode array/scheme may be comprised of placement of the studied foodstuff within a generated electrical field array, on an electrode scheme array, placing the electrode array about around or as comprised in such configuration as to measure ‘capture’, characterize and illustrate the unique geometry and traits of the subject foodstuff in its entirety or as possible the electrode scheme and array may be introduced directly into the study subject foodstuff, and/or that such electrical power management configurations may be comprised of fixed or variable frequencies, currents and voltages and circuit models (series and/or parallel) and that the measured and calculated values be comprised of such values and sampling rates to adequately capture, characterize and illustrate the unique geometry and traits of the subject foodstuff in its entirety.

The electrical signals utilized to measure and calculate the impedance, resistance, reactance, capacitance and phase angle may be comprised of multiple schemes based upon the type and geometry of the foodstuff; a mono or singular frequency, multiple frequencies, or a spectroscopic illustration across a segment or band of frequencies.

The measured and calculated electrical values comprised of impedance, resistance, reactance, capacitance and phase angle are related to the comprised physiological values of fluid; volume and distribution, the cell mass; volume, character and membrane vitality as related to the unique and inherent characteristics palatability (flavor, juiciness and tenderness) of the studied subject foodstuff and reported in such a manner as to provide a basis for objective assessments and subjective interpretation of the comprised values for foodstuff product; safety grading, pricing, handling, management and disposition.

Thus, the present invention provides a method and system for the use of bioelectrical impedance analysis (BIA) in the electrical measurement of a biological equivalent model of ‘live’ foodstuffs or ‘biological entities’ to provide an objective assessment and scale of safety and freshness as related to the characteristics, volume and distribution of fluids, tissues and cells as well as the electrical vitality of cells and cell membranes through the measurement of impedance (Z), resistance (R), reactance (Xc), capacitance (Cp) and the calculation of phase angle (Pa) at a fixed or variable electrical frequency, current and voltage through a tetrapolar electrode scheme placed on, around and/or in or with the subject placed upon the arrayor by placing the study subject within a electrical field or a portion thereof by placing the foodstuff biological entity or a portion thereof onto an electrode configuration singularly or as comprised as part of an external appliance; such as part of a scale; refrigerator or a portable temperature controlling device, packaging or display, the study subject as measured individually; compared to normal, average and optimal values and as tracked serially over time and compared to changes from the initial measurement.

More specifically the present invention provides a method and system for determining the palatability of a portion or whole live or previously live foodstuff such as a meat, fish, fowl, fruit or vegetable, to grade its characteristics (consumption safety), quality and salability, and to support decisions regarding its disposition, preparation and presentation and cost and consumption.

The methods of the present invention can utilize a modification of the body composition analyzer disclosed in U.S. Pat. No. 5,372,141, the entire contents of which are incorporated herein by reference thereto. Such modification may include, but not be limited to, impedance measuring instrumentation capable of measuring impedance, resistance and reactance for the calculation of capacitance and phase angle from selected singular or mono-frequency, multiple frequencies and/or impedance spectroscopic analysis or changes in current, power and voltage.

In accordance with the present invention, utilization of BIA in a biological model provides an objective assessment of the study subject's (whole or section of the biological entity) volume and distribution of fluids, tissues and cells, as well as the electrical health and vitality of the cells and membranes.

The characteristics of BIA include precision, accuracy, feasibility and economy. BIA may be applied to any subject whole or an area of representative sample or interest to be studied and examined for safety for consumption; a section thereof, regionally, or to the whole biological entity. It is non-offensive, causing no harm. It may be repeated freely, as desired to capture various dynamic changes unique to the variety of live foodstuffs (biological entities), to illustrate initial values and change over time so that progression of conditions can be monitored and changes that affect consumption safety determined. The specific value of BIA is in its precision of measurement and the significance of the electrically measured products illustration of the biological foodstuff entities equivalent physiological variables of fluid, tissue and cells volume and distribution, cell membrane volume and vitality, derivative values initially and comparison to average, optimal, normal, and subsequent individual values and changes serially over time.

Based upon the individual genus, type; species, ‘cut’ or sample of the biological foodstuff entity, palatability is determined by the baseline values, and changes thereto (rate, zenith and nadir) of the measured and calculated values initially and over time. The properties of the electrical values directly relate to biological equivalents such as; measured R is inverse to water content (juiciness) so an increasing R value is indicative of water loss and a decreasing R value is indicative of water accumulation as well as measured Xc is proportional to cell mass so a decreased Xc is indicative of cell membrane loss through such processes (naturally occurring or artificially induced) as fragmentation or proteolysis; a diminution of the Xc value and/or a change in the rate of the diminution from a zenith towards a nadir is indicative of optimal safety for consumption which may progress beyond that nadir of safety and become non-safe.

Comparison of the Xc value of one sample of the same genus and species, section and cut of a biological entity to another sample of the same genus and species, section and cut of a biological entity illustrates a comparative scale of consumption safety.

The invention provides a process of selecting a preparation method, a packaging and shipping method, or disposition of a foodstuff, comprising the steps of: first determining a palatability value for the foodstuff; and second determining as a function of the determined palatability value whether or not the foodstuff is to be prepared for consumption, is to be packaged and shipped, or is to be disposed of.

The process may include the steps of: determining a first palatability index of the foodstuff at harvesting the foodstuff or as close in time as possible to the harvesting of the foodstuff; determining a second palatability index of the foodstuff just prior to the second determining step or as close in time as possible to performing the second determining step; and determining the palatability value as a function of the first and second palatability indices.

The process may include the step of: selecting a method of preparation for the foodstuff in dependence on the determined palatability value.

The process may include the step of selecting a method of preparation for the foodstuff in dependence on the determined palatability value.

The process may include the step of preparing the foodstuff in accordance with the selected method to optimize the determined palatability value.

The process may include selecting of a method of preparation for the foodstuff in dependence on the determined palatability value comprises grading of the determined palatability value for preparation of the foodstuff as an entree rather than as a part of a sauced, casserole, stew and/or secondary or supporting portion of a meal.

The process may also entail using the determined palatability value as a means to determine packaging type and transportation rate in that at the point of assessment the foodstuff is sorted by palatability with application and value in mind, and then packed and sent to distribution or end-user points based upon the determined palatability value.

The first determining step includes subjecting the foodstuff to bioelectrical impedance analysis including measurement and/or calculation of values of resistance, reactance, impedance, capacitance, and/or phase angle of the foodstuff.

The process may include utilizing results of the subjecting step to illustrate whether or not the foodstuff has been previously frozen or is fresh.

Determining of the first and second palatability indices includes subjecting the foodstuff to bioelectrical impedance analysis including measurement and/or calculation of values of resistance, reactance, impedance, capacitance, and/or phase angle of the foodstuff.

The invention provides a process of determining whether or not a foodstuff is fresh or has previously been frozen, comprising the steps of: subjecting the foodstuff to bioelectrical impedance analysis including measurement and/or calculation of values of resistance, reactance, impedance, capacitance, and/or phase angle of the foodstuff; and utilizing results of the subjecting step to illustrate whether or not the foodstuff has been previously frozen or is fresh.

When the reactance value is zero or nearly zero, this indicates that the foodstuff has previously been frozen.

The phase angle may be used to indicate whether or not the foodstuff has been previously frozen or is fresh.

Specific applications of the measured impedance values in foodstuffs include, fresh v frozen, meal positioning and preparation method as follows.

First, the determination of fresh versus frozen involves a determination made by measuring an apparently-fresh foodstuff specimen

If the reactance value of the foodstuff specimen is zero or nearly zero, then this indicates that the cell membranes have been obliterated by freezing (because it obviously was not cooked, as cooking visibly alters the foodstuff).

Secondly, the grading of a foodstuff's palatability for preparation as an entrée, rather than as a part of a sauced, casserole, stew and/or secondary or supporting portion of a meal, is undertaken.

For instance, a piece of fish which has a measured phase angle (palatability index) closer to the average value found at the time of harvesting that fish (saltwater fish ˜40°; freshwater fish 35° but >20°) will have attributes of palatability suitable for an entrée.

However, if the phase angle value/palatability index is <20° but >5°, then the fish portion is not suitable as an entrée (stand-alone), but is better masked within a dominant sauce or covering.

Meats, particularly beef, are different in that upon harvest the palatability index/phase angle value illustrates improved palatability attributes as the specimen ages (dry or wet) and while the value upon beef harvest may range from 45° to 50° with the lower value indicative of increased tenderness the overall palatability index improves as the value decreases.

This decreasing value is used as an objective numeric value associated with a subjective personal taste preference as well as correlating the value to a preparation method.

For example, some people prefer NY Strip steak grilled at an index level of between 25° to 30°, broiled from 20° to 25°, and blackened with spices from 15° to 20°

In fruits and vegetables, palatability and nutritive content diminish rapidly over time from harvest, extended translocation, improper handling, and growth method.

Comparative analysis of initial versus subsequent measured values with the least decrease in the palatability index (Phase Angle) is associated with the better (more palatable) specimen.

There have been described hereinabove several of the unique and novel embodiments of the present invention which can be practiced in many different ways.

Many changes, modifications, variations, and other uses and applications will become apparent to those persons skilled in this particular area of technology and to others after having been exposed to the present patent application.

Any and all such changes, modifications, variations and other uses and applications which do not depart from the spirit and scope of the present invention are therefore covered by and embraced within the present invention and the patent claims set forth below.

1. A process of selecting a preparation method, a packaging and shipping method, or disposition of a foodstuff, comprising the steps of:
first determining a palatability value for the foodstuff; and
second determining as a function of the determined palatability value whether or not the foodstuff is to be prepared for consumption, is to be packaged and shipped, or is to be disposed of.

2. The process of claim 1, including the steps of:
determining a first palatability index of the foodstuff at harvesting the foodstuff or as close in time as possible to the harvesting of the foodstuff;
determining a second palatability index of the foodstuff just prior to the second determining step or as close in time as possible to performing the second determining step; and
determining the palatability value as a function of the first and second palatability indices.

3. The process of claim 1, including the step of:
selecting a method of preparation for the foodstuff in dependence on the determined palatability value.

4. The process of claim 2, including the step of:
selecting a method of preparation for the foodstuff in dependence on the determined palatability value.

5. The process of claim 3, including the step of:
preparing the foodstuff in accordance with the selected method to optimize the determined palatability value.

6. The process of claim 4, including the step of:
preparing the foodstuff in accordance with the selected method to optimize the determined palatability value.

7. The process of claim 3, including the steps of:
determining a first palatability index of the foodstuff at harvesting the foodstuff or as close in time as possible to the harvesting of the foodstuff;
determining a second palatability index of the foodstuff just prior to the selecting step or as close in time as possible to performing the selecting step; and
determining the palatability value as a function of the first and second palatability indices.

8. The process of claim 5, including the steps of:
determining a first palatability index of the foodstuff at harvesting the foodstuff or as close in time as possible to the harvesting of the foodstuff;
determining a second palatability index of the foodstuff just prior to the selecting step or as close in time as possible to performing the selecting step; and
determining the palatability value as a function of the first and second palatability indices.

9. The process of claim 3, wherein:
the selecting of a method of preparation for the foodstuff in dependence on the determined palatability value comprises grading of the determined palatability value for preparation of the foodstuff as an entree rather than as a part of a sauced, casserole, stew and/or secondary or supporting portion of a meal.

10. The process of claim 1, including the step of:
using the determined palatability value as a means to determine packaging type and transportation rate in that at the point of assessment the foodstuff is sorted by palatability with application and value in mind, and then packed and sent to distribution or end-user points based upon the determined palatability value.

11. The process of claim 1, wherein:
the first determining step includes subjecting the foodstuff to bioelectrical impedance analysis including measurement and/or calculation of values of resistance, reactance, impedance, capacitance, and/or phase angle of the foodstuff.

12. The process of claim 11, including the step of:
utilizing results of the subjecting step to illustrate whether or not the foodstuff has been previously frozen or is fresh.

13. The process of claim 12, wherein:
when the reactance value is zero or nearly zero, this indicates that the foodstuff has previously been frozen.

14. The process of claim 12, wherein;
the phase angle is used to indicate whether or not the foodstuff has been previously frozen or is fresh.

15. The process of claim 2, wherein:
the determining of the first and second palatability indices includes subjecting the foodstuff to bioelectrical impedance analysis including measurement and/or calculation of values of resistance, reactance, impedance, capacitance, and/or phase angle of the foodstuff.

16. The process of claim 15, including the step of:
utilizing results of the subjecting step to illustrate whether or not the foodstuff has been previously frozen or is fresh.

17. The process of claim 16, wherein:
when the reactance value is zero or nearly zero, this indicates that the foodstuff has previously been frozen.

18. A process of determining whether or not a foodstuff is fresh or has previously been frozen, comprising the steps of:
subjecting the foodstuff to bioelectrical impedance analysis including measurement and/or calculation of values of resistance, reactance, impedance, capacitance, and/or phase angle of the foodstuff; and
utilizing results of the subjecting step to illustrate whether or not the foodstuff has been previously frozen or is fresh.

19. The process of claim 18, wherein;
the phase angle is used to indicate whether or not the foodstuff has been previously frozen or is fresh.

20. The process of claim 18, wherein:
when the reactance value is zero or nearly zero, this indicates that the foodstuff has previously been frozen.
